(19) United States
(12) Patent Application Publication
(10) Pub. No.: US 2026/0224284 A1
(43) Pub. Date: Aug. 6, 2026

(54) METHOD FOR PROVIDING IMPLANT IMPLANTATION POSITION AND DEVICE THEREFOR

(71) Applicant: OSSTEM IMPLANT CO., LTD., Seoul (KR)

(72) Inventors: Kyoo Ok CHOI, Seoul (KR); Sang Hyeong CHO, Gimpo-si (KR)

(73) Assignee: OSSTEM IMPLANT CO., LTD., Seoul (KR)

(21) Appl. No.: 19/150,720

(22) PCT Filed: Jan. 15, 2024
(86) PCT No.: PCT/KR2024/000677

§ 371 Date: Jul. 24, 2025

(30) Foreign Application Priority Data
Mar. 15, 2023 (KR) 10-2023-0034204

(51) Int. Cl. A61B 34/10; A61C 1/08

(52) U.S. Cl. CPC A61B 34/10; A61C 1/084; A61B 2034/102; A61B 2034/107

(57) ABSTRACT

Disclosed are a method for providing an implant implantation position and a device therefor. The method for providing an implant implantation position and the device therefor according to an embodiment match, by photographing an oral cavity of a patient, surgery data including a radiopaque tool and a surgical guide and surgery data of the patient, which has been used for a surgery planning process, so as to identify whether an actual surgery guide is accurately fastened within the oral cavity of the patient, and provide an expected implantation position of the implant based on the identification so as to identify a deviation from the planned implant.

### TECHNICAL FIELD

The present invention relates to dental image processing technology, and more specifically, to image processing and user interface technology for guided dental implant surgery.

### BACKGROUND ART

When performing dental implant surgery, an implant surgery plan is established in advance using software. The process of establishing a dental implant surgery plan using software generally involves acquiring computerized tomography (CT) data and dental data of a patient, matching the data, establishing a surgery plan that determines the positions of implant structures (e.g., crown, implant, abutment, anchor pin, etc.) in consideration of the patient's anatomical structure, and then designing a surgical guide shape based on the plan information.

During the design of the surgical guide shape using software, the patient's dental data is loaded into the software and used as a reference for designing the guide shape. However, if an error occurs during the process of acquiring the patient's dental data, or if an error occurs during the process of outputting the designed guide using a three-dimensional (3D) printer, a surgical guide with such error may be fastened within the oral cavity of the patient, causing the implant to be placed at a position different from the planned position.

Some commercial software provides a function that compares the planned implant position with the actual implant position after surgery, based on CT data. However, once the surgery has already been completed, it is often difficult to correct the implant position where an error has occurred.

### DISCLOSURE

### Technical Problem

According to an embodiment, a method for providing an implant placement position and a device therefor are proposed, which enable identification of whether an actual surgical guide for implant surgery is accurately fastened within the oral cavity of a patient.

In addition, a method for providing an implant placement position and a device therefor are proposed, which enable identification of a deviation from the planned implant position before surgery by providing an expected placement position of the implant before surgery using a radiopaque tool.

### Technical Solution

According to an embodiment, a method for providing an implant placement position may include the steps of: acquiring, by a device for providing an implant placement position, pre-surgery data of a patient including design of a virtual guide shape to be used for surgical planning; acquiring surgery data including a radiopaque tool and a surgical guide by photographing an oral cavity of the patient; matching the surgery data to the pre-surgery data; generating a virtual implant expected to be placed on the basis of a radiopaque tool in the matched surgery data; and providing placement position information of an implant expected to be placed.

The method may further include the step of determining, by the device for providing an implant placement position, a placement position of an object including the virtual implant from the pre-surgery data of the patient and determining a position of a drilling hole based on the determined placement position of the implant, followed by designing and outputting a virtual guide shape including the determined drilling hole.

In the step of acquiring the surgery data, the device for providing an implant placement position may fasten an actual radiopaque tool into a drilling hole of an actual surgical guide that is mounted in the oral cavity of the patient, and then photograph the oral cavity to acquire the surgery data, and in the step of matching the surgery data, the device for providing an implant placement position may match the surgery data on the basis of the pre-surgery data by adjusting the position and orientation of the surgery data to align with the position and orientation of the pre-surgery data.

The method may further include the step of, after the device for providing an implant placement position matches the pre-surgery data and the surgery data, performing fine-adjustment of a matching result using a manipulator.

The method may further include the step of, after the device for providing an implant placement position matches the pre-surgery data and the surgery data, matching a radiopaque tool provided from a software library on the basis of the radiopaque tool represented in the matched surgery data.

The method may further include the step of selecting, by the device for providing an implant placement position, a radiopaque tool from the library to be matched with the radiopaque tool of the surgery data, wherein the device for providing an implant placement position may select the type of radiopaque tool based on a specification of an implant planned before surgery for each tooth number.

In the step of matching the radiopaque tools, the device for providing an implant placement position may match the radiopaque tools by adjusting the position and direction of the radiopaque tool provided from the software library with respect to the radiopaque tool represent in the matched surgery data.

In the step of matching the radiopaque tools, the device for providing an implant placement position may extract and match points for the two radiopaque tools, the points including a topmost center point, a point on a stopper extending across a drilling hole of the surgical guide, and a corresponding point on an opposite side of the stopper.

The method may further include the step of providing, by the device for providing an implant placement position, a matching result indicating whether a shape of the radiopaque tool in the matched surgery data matches a margin line of the radiopaque tool provided from the software library, as an image screen.

In the step of providing the matching result as the image screen, the device for providing an implant placement position may provide the matching result while varying a position at which a cross-section is generated within a two-dimensional (2D) cross-sectional image screen that provides the matching result between the two radiopaque tools.

The method may further include the step of providing, by the device for providing an implant placement position, an image screen comparing the position and direction of an implant planned using the pre-surgery data with those of the virtual implant expected to be placed in a state in which an actual surgical guide is fastened in the patient's oral cavity.

In the step of providing the image screen, the device for providing an implant placement position may measure an angle between a reference axis of the virtual implant expected to be placed and a reference axis of the implant planned before surgery, and provide an angle difference, wherein when the angle difference between the reference axis of the implant planned before surgery and the reference axis of the virtual implant expected to be placed is greater than a preset angle, the device may determine that an error has occurred, and provide error information.

In the step of providing the image screen, a measurement interface that allows the user to check the error information may be provided.

In the step of providing the image screen, the device for providing an implant placement position may measure an angle between a reference axis of the implant planned before surgery and a reference axis of the virtual implant expected to be placed and provide angle deviation information.

In the step of providing the image screen, when the virtual implant expected to be placed deviates from a safety region of the implant planned before surgery, the device for providing an implant placement position may provide warning information indicating this deviation.

According to another embodiment, a device for providing an implant placement position may include a data acquisition unit configured to acquire pre-surgery data of a patient including design of a virtual guide shape to be used for surgical planning, and acquire surgery data including a radiopaque tool and a surgical guide by photographing an oral cavity of the patient; a control unit configured to match the surgery data to the pre-surgery data and generate a virtual implant expected to be placed on the basis of a radiopaque tool in the matched surgery data; and an output unit configured to output placement position information of an implant expected to be placed.

The control unit may display, via the output unit, an image screen comparing positions and directions between an implant planned using the pre-surgery data and the virtual implant expected to be placed in a state in which the patient is wearing an actual surgical guide

After matching the pre-surgery data and the surgery data, the control unit may match a radiopaque tool provided from a software library on the basis of the radiopaque tool represented in the matched surgical data.

### Effects of the Invention

According to a method and device for providing an implant placement position in accordance with an embodiment, it is possible to identify whether an actual surgical guide for implant surgery is accurately fastened within the oral cavity of a patient.

In addition, by providing an expected placement position of the implant using a radiopaque tool, it is possible to identify a deviation from the planned implant position before surgery.

Furthermore, an operator is allowed to predict the result of the guided implant surgery in advance based on the expected placement position of the implant, thereby improving the accuracy of the guided surgery.

### DESCRIPTION OF DRAWINGS

FIG. 1 is a diagram illustrating a configuration of a device for providing an implant placement position according to an embodiment of the present invention.

FIG. 2 is a diagram showing a screen for generating a drilling hole based on an implant in first pre-surgery data according to an embodiment of the present invention.

FIG. 3 is a diagram showing a screen for generating a drilling hole and a guide shape in second pre-surgery data according to an embodiment of the present invention.

FIG. 4 is a diagram showing surgery data obtained by imaging a state in which an actual surgical guide including an actual radiopaque tool is fastened within the oral cavity of a patient according to an embodiment of the present invention.

FIG. 5 is a drawing showing the shapes of radiopaque tools by specification according to one embodiment of the present invention.

FIG. 6 is a diagram showing pre-surgery data and surgery data of a patient, which are subjects for matching, in a three-dimensional form, according to an embodiment of the present invention.

FIG. 7 is a diagram showing an example of matching points between a radiopaque tool in surgery data and a radiopaque tool from a software library according to an embodiment of the present invention.

FIG. 8 is a diagram showing an image screen that provides a matching result of the radiopaque tool on the surgery data according to an embodiment of the present invention.

FIG. 9 is a diagram showing an image screen that provides a matching result of the radiopaque tool using a margin line according to an embodiment of the present invention.

FIG. 10 is a diagram showing a screen for generating a virtual implant expected to be placed on the basis of the radiopaque tool according to an embodiment of the present invention.

FIG. 11 is a diagram showing (a) an image screen of an implant planned before surgery, (b) an image screen of a virtual implant expected to be placed based on surgery data, and (c) an image screen in which both implants are superimposed, according to an embodiment of the present invention.

FIG. 12 is a drawing showing an image screen that provides error information between a position of an implant planned before surgery and a position of a virtual implant expected to be placed according to one embodiment of the present invention.

FIG. 13 is a diagram showing an image screen that provides warning information when the virtual implant expected to be placed deviates from a safety region of the implant planned before surgery, according to an embodiment of the present invention.

FIG. 14 is a flowchart illustrating a method for providing an implant placement position according to an embodiment of the present invention.

### MODE OF THE INVENTION

The advantages and features of the present invention and the manner of achieving the advantages and features will become apparent with reference to embodiments described in detail below together with the accompanying drawings. However, the present invention may be implemented in many different forms and should not be construed as being limited to the embodiments set forth herein, and the embodiments are provided such that this disclosure will be thorough and complete and will fully convey the scope of the present invention to those skilled in the art, and the present invention is defined only by the scope of the appended claims. Throughout the entire specification, the same or like reference numerals designate the same or like elements.

In describing the example embodiments, a detailed description of related known configurations or functions incorporated herein will be omitted when it is determined that the detailed description thereof may unnecessarily obscure the subject matter of the present invention. The terms which will be described below are terms defined in consideration of the functions in the present invention, and may be different according to users, intentions of the users, or customs. Therefore, the definitions of the terms used herein should follow contexts disclosed herein.

Hereinafter, example embodiments of the present invention will be described in detail with reference to the accompanying drawings. However, the example embodiments may be modified in various different forms, and the scope of the present invention is not limited to the example embodiments described below. The embodiments of the present invention are provided to aid those skilled in the art in the explanation and the understanding of the present invention.

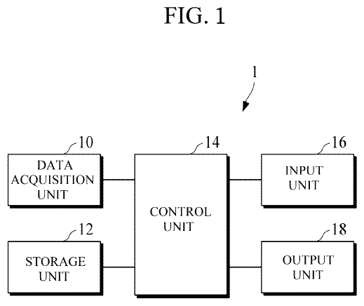

FIG. 1 is a diagram illustrating a configuration of a device for providing an implant placement position according to an embodiment of the present invention.

Referring to FIG. 1, a device 1 for providing an implant placement position is an electronic device capable of executing a medical image processing program. The electronic device may include a personal computer (PC), a notebook computer, a laptop computer, a tablet computer, a smartphone, a mobile phone, or the like. Examples of the medical image processing program include a guide design program, a scanning program, a CAD program, and the like. In addition, the program may be applied not only to dental implant surgery but also to general medical image processing applications. Hereinafter, for convenience of description, a guide design program for dental implant surgery will be described as an example. However, it should be understood that the invention is equally applicable to other programs as long as image processing is possible.

The image processing procedure using the medical image processing program includes: registering a surgery patient; acquiring dental data of the registered patient; generating an arch line from the dental data and creating a panoramic image using the arch line; setting the position of a crown model in the dental data; setting a placement position of the implant and a guide area in the dental data; and generating and outputting a guide.

The device 1 for determining an implant placement position according to an embodiment includes a data acquisition unit 10, a storage unit 12, a control unit 14, an input unit 16, and an output unit 18.

The data acquisition unit 10 acquires image data. For example, the data acquisition unit 10 may acquire pre-surgery data and surgery data through a linked medical imaging device (not shown) or a separate database, etc. The pre-surgery data refers to data that can be acquired before an actual surgical guide is fastened to the patient, and it is obtained from the patient's oral cavity including a damaged target tooth. For example, the pre-surgery data may include computerized tomography (CT) data, scan data, and may also include intraoral model data of the patient.

The control unit 14 may implant a virtual object according to a surgery plan established based on the pre-surgery data and may design a guide shape for implanting the virtual object. The object may include, for example, a crown, an implant, an abutment, an anchor pin, and the like.

The output unit 18 may output a guide shape designed by the control unit 14 to a linked 3D printer or milling device. Here, the designed guide shape may be in a data format compatible with the 3D printer or milling device, and the 3D printer or milling device may output a physical guide based on the input guide shape. Hereinafter, the output guide shape is referred to as a “surgical guide.”

The data acquisition unit 10 acquires surgery data obtained by imaging a state in which a surgical guide combined with a radiopaque tool is fastened within the oral cavity of the patient. For example, the patient may bite the surgical guide, and a CT image may be captured using a CT imaging device. The radiopaque tool serves as a type of marker and may appear in the CT image obtained through the CT imaging.

The pre-surgery data is data obtained from the patient's oral cavity before the surgery planning process for the purpose of designing the guide shape, whereas the surgery data is data obtained from the patient's oral cavity after the surgical guide is actually fastened therein. These two types of data are thus distinguishable.

The storage unit 12 stores various types of data, including information necessary for operating the device 1 for determining an implant placement position and information generated during operation. For example, the storage unit 12 may store pre-surgery data and surgery data for individual patients and provide the data to the control unit 14 upon user request.

The control unit 14 may match the surgery data, which is obtained by imaging a state in which an actual surgical guide combined with an actual radiopaque tool is fastened within the patient's oral cavity, to the pre-surgery data used for surgery planning, so as to identify whether the actual surgical guide has been accurately fastened within the patient's oral cavity. Specifically, the control unit 14 performs matching between the surgery data and the pre-surgery data, using the pre-surgery data as a reference. For example, the control unit 14 may perform matching on the basis of the pre-surgery data by adjusting the position and orientation of the surgery data to align with the position and orientation of the pre-surgery data. For matching between two pieces of data, the control unit 14 may select the patient's tooth shapes or anatomical structures from each data as matching points. Hereinafter, the data in which the surgery data is matched to the pre-surgery data is referred to as “matched surgery data.”

The control unit 14 may match a radiopaque tool provided from a software library to the radiopaque tool in the matched surgery data.

The software library is a database containing information about surgical guides, and may include information such as the shape, size, image, and material of the surgical guide, as well as the shape, size, and position of the radiopaque tool mounted on the corresponding surgical guide. For matching between radiopaque tools, the control unit 14 may identify information about the surgical guide worn by the patient in the matched surgery data, or when the information about the surgical guide is identified through user input, the control unit 14 may load the radiopaque tool corresponding to the surgical guide from the software library.

The control unit 14 may display, on a screen of a display device linked via the output unit 18, a matching result between the radiopaque tool shape in the matched surgery data and the radiopaque tool provided from the software library. For example, the control unit 14 may provide, as an image screen via the output unit 18, a matching result indicating whether the margin line of the radiopaque tool in the matched surgery data matches the margin line of the radiopaque tool from the software library.

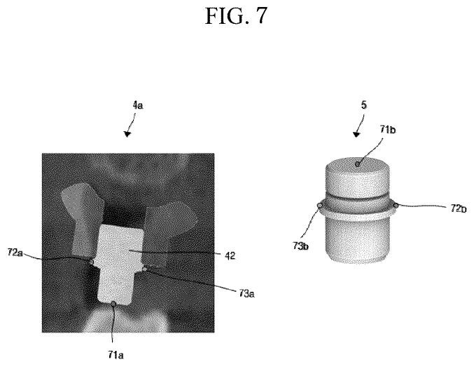

The matching process between the radiopaque tools described above may proceed depending on whether an error has occurred in the radiopaque tool on the surgery data. For example, the control unit 14 may determine that an error has occurred when distortion occurs in the radiopaque tool or the shape of the radiopaque tool is not fully represented during the image acquisition process. An embodiment related to this will be described below with reference to FIG. 7.

Subsequently, the control unit 14 may generate the shape of a virtual implant expected to be placed, based on the matched radiopaque tool, and may provide the guide suitability result and the placement position information of the implant expected to be placed to the output unit 18, and the output unit 18 may then output them.

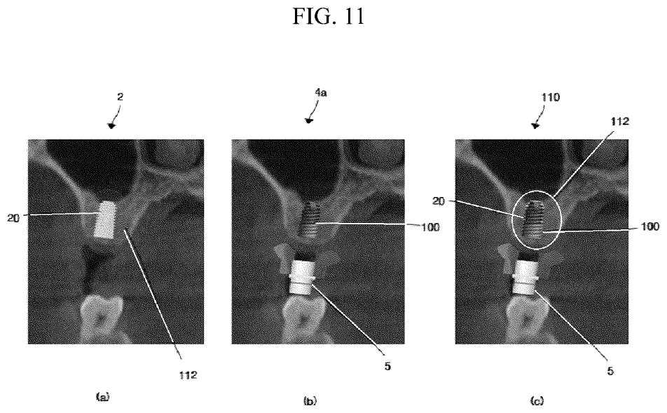

Here, when the placement position information of the virtual implant is expected to lie within a safety region based on the position information of the planned implant, the control unit 14 may output, via the output unit 18, an indication or guidance that the guide is suitable for the patient's oral cavity. Furthermore, the control unit 14 may provide, via the output unit 18, an image screen that compares the positions and directions between the implant planned using the pre-surgery data and the virtual implant expected to be placed in the state in which the actual surgical guide is fastened in the patient's oral cavity. An embodiment related to this will be described below with reference to FIG. 11.

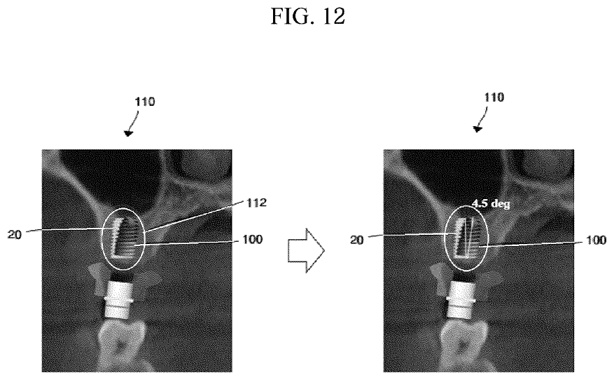

For example, in a superimposed image screen where the planned implant and the virtual implant are overlapped, the control unit 14 may provide, via the output unit 18, error information indicating how much the position of the virtual implant expected to be placed deviates from the position of the implant planned before surgery. In this case, the control unit 14 may provide, via the output unit 18, a measurement interface that allows the user to check the error information between the position of the implant planned before surgery and the position of the virtual implant expected to be placed. An embodiment related to this will be described below with reference to FIG. 12.

As another example, the control unit 14 may measure the angle between a reference axis of the virtual implant expected to be placed and a reference axis of the implant planned before surgery, and provide the angle difference via the output unit 18. In this case, when the angular difference between the reference axis of the planned implant and that of the virtual implant exceeds a preset angle, the control unit 14 may determine that an error has occurred and provide the error information via the output unit 18. An embodiment related to this will be described below with reference to FIG. 12.

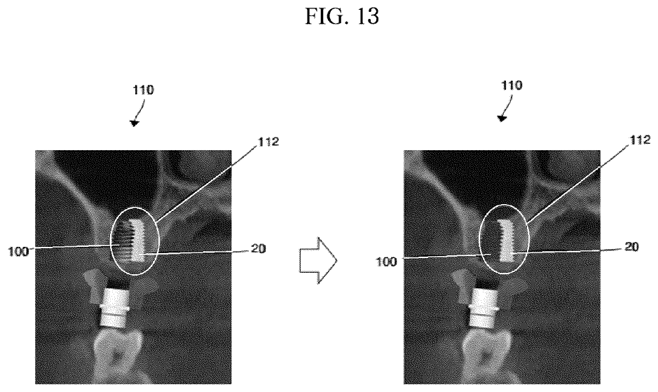

As yet another example, when the virtual implant expected to be placed deviates from the safety region of the implant planned before surgery, the control unit 14 may provide warning information indicating this, via the output unit 18. An embodiment related to this will be described below with reference to FIG. 13.

The input unit 16 receives a user operation signal.

The output unit 18 displays various types of information on a screen and may output the guide shape designed by the control unit 14 to the linked 3D printer or milling machine.

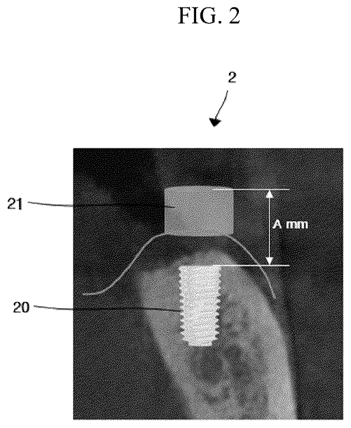

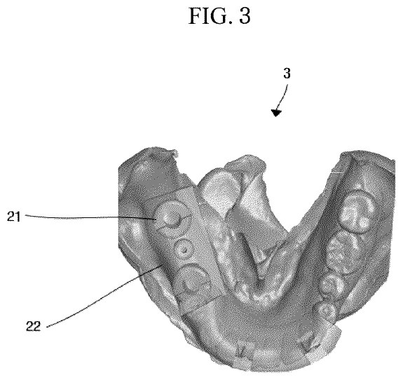

FIG. 2 is a diagram showing a screen for generating a drilling hole based on an implant in first pre-surgery data according to an embodiment of the present invention, and FIG. 3 is a diagram showing a screen for generating a drilling hole and a guide shape in second pre-surgery data according to an embodiment of the present invention.

Referring to FIGS. 1 to 3, the device 1 for providing an implant placement position determines a placement position of a virtual implant 20 in pre-surgery data 2 of a patient, determines the position of a drilling hole 21 based on the determined placement position of the implant 20, and then designs a virtual guide shape 22 including the determined drilling hole 21 and a predetermined tooth. For example, as shown in FIG. 2, the device 1 for determining an implant placement position may acquire first pre-surgery data 2 (e.g., CT data) of the patient, determine the placement position of the implant 20, and determine the position of the drilling hole 21. In addition, as shown in FIG. 3, the device 1 may acquire second pre-surgery data 3 (e.g., intraoral model data) and design a guide shape 22 including the drilling hole 21.

The device 1 for determining an implant placement position may determine the placement position of the implant 20 through manipulation of the position and orientation of the implant 20 by the user. In another example, the device 1 may acquire anatomical structure information such as crown information and adjacent tooth information using an algorithm or artificial intelligence (AI) and automatically determine an initial position of the implant 20. Thereafter, the device 1 may determine a final placement position of the implant 20 by allowing the user to adjust the initial position of the implant.

In this case, the user may adjust the position using a manipulator provided by the device 1 for determining an implant placement position.

Once the placement position of the implant 20 is determined, the device 1 for determining an implant placement position may, as shown in FIG. 2, generate the drilling hole 21 in a region spaced by a certain distance (A mm) from a first position of the implant 20 (e.g., the uppermost point of the implant) to a second position of the drilling hole 21 (e.g., the uppermost point of the drilling hole) in the first pre-surgery data 2 (e.g., CT data), based on the reference axis of the implant 20.

Subsequently, as shown in FIG. 3, the device 1 for determining an implant placement position may design the guide shape 22 including the drilling hole 21 based on the second pre-surgery data (e.g., oral model data) 3. For example, in the process of designing the guide shape 22, the device 1 may generate the guide shape 22 in a manual mode by receiving, from the user, input of point locations along a line where the guide shape is to be generated. In another example, the device 1 for determining an implant placement position may automatically generate an initial guide shape using an algorithm or AI, such that a certain number of teeth adjacent to the drilling hole 21 are included. When the user inputs an adjustment signal for the initial guide shape via the provided manipulator, the device may generate a final guide shape in which the adjustment signal is applied.

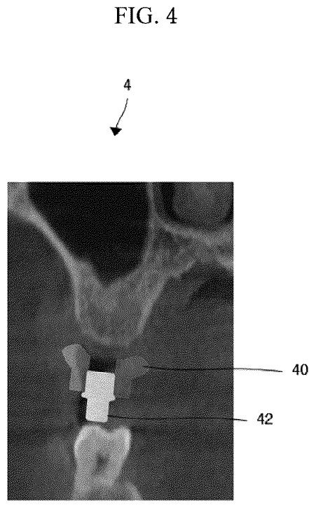

FIG. 4 is a diagram showing surgery data obtained by imaging a state in which an actual surgical guide including an actual radiopaque tool is fastened within the oral cavity of a patient, according to an embodiment of the present invention.

Referring to FIGS. 1 and 4, the device 1 for determining an implant placement position provides a surgical guide by outputting the completed guide shape using a 3D printer or milling equipment.

When an operator combines an actual radiopaque tool that matches the specifications of the drilling hole of the actual surgical guide with the drilling hole of the surgical guide and fastens it in the patient's oral cavity, the device 1 for determining an implant placement position acquires surgery data 4 by imaging this state. That is, by imaging the patient's oral cavity, surgery data 4 including the surgical guide in which the radiopaque tool is mounted may be obtained. As shown in FIG. 4, the surgery data 4 of the patient acquired in this way includes a surgical guide 40 with a radiopaque tool 42 combined therewith, allowing the operator to visually confirm the shape of the radiopaque tool 42 in the image.

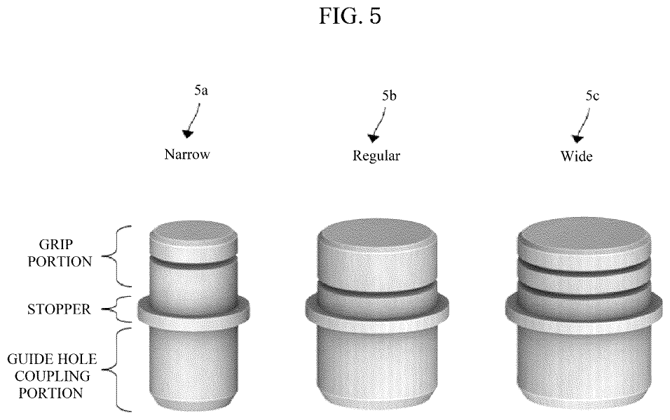

FIG. 5 is a diagram illustrating the shapes of radiopaque tools by specification, according to an embodiment of the present invention.

Referring to FIGS. 1 and 5, the drilling hole size of the surgical guide may vary by manufacturer depending on the implant specifications. For example, implant sizes may be classified into three categories: narrow, regular, and wide. In the case of a narrow implant, the diameter may be φ3.0 or less, and the drilling hole diameter may be φ3.6. In the case of a regular implant, the diameter may be greater than φ3.0 and φ4.5 or less, and the drilling hole diameter may be φ5.1. In the case of a wide implant, the diameter may be φ5.0 or more, and the drilling hole diameter may be φ5.8.

Accordingly, as shown in FIG. 5, radiopaque tools 5a, 5b, and 5c corresponding to the sizes of the respective drilling holes may be used. Each of the radiopaque tools 5a, 5b, and 5c is composed of a grip portion, a stopper, and a guide hole coupling portion.

Although the diameter sizes of the radiopaque tools 5a, 5b, and 5c differ so that they can be inserted into their respective drilling holes of the surgical guide, the device 1 for determining an implant placement position may provide markers on the radiopaque tools to facilitate user identification. For example, the device 1 may label each radiopaque tool with a number (marking). As shown in FIG. 5, each radiopaque tool 5a, 5b, and 5c includes a stopper that limits the portion inserted into the drilling hole of the surgical guide for each size, and the user may insert each radiopaque tool 5a, 5b, or 5c into the drilling hole until the insertion is blocked by the stopper.

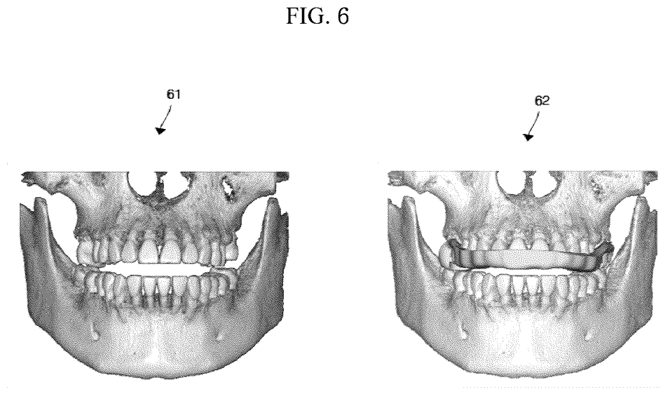

FIG. 6 is a diagram showing pre-surgery data 61 and surgery data 62 of a patient, which are subjects for matching, in a three-dimensional form, according to an embodiment of the present invention.

Referring to FIGS. 1 and 6, the device 1 for determining an implant placement position matches the patient's pre-surgery data 61 and surgery data 62, as shown in FIG. 6, in order to compare the position and orientation of the implant as planned before surgery (i.e., the position and orientation of the implant as planned on the pre-surgery data) with those of the implant expected to be placed during the surgical process (i.e., the position and orientation of the virtual implant generated in the surgery data).

The device 1 for providing an implant placement position may automatically match the surgery data 62 to the pre-surgery data 61, and AI may be used for this purpose. For example, the device 1 may trace tooth shapes or anatomical structures in the two pieces of data 61 and 62 using training data, and perform matching by aligning the coordinates of the two pieces of data 61 and 62 based on the traced tooth shapes or anatomical structures.

If the accuracy of the matching result obtained using AI is insufficient (for example, if the coordinates of the two pieces of data 61 and 62 do not align), the device 1 for providing an implant placement position may perform matching through a semi-automatic method in which a certain number of points (e.g., three points) are input to each of the two pieces of data 61 and 62 by a user operation signal, so that the coordinates of the data 61 and 62 match each other. In both automatic and semi-automatic matching, the user may check the matching result and, if necessary, modify it using a matching adjustment manipulator.

Since the implant planned before surgery has its position determined on the pre-surgery data 61, the device 1 for providing an implant placement position may acquire the position information and shape of the implant planned before surgery from the pre-surgery data 61. Because the implant planned before surgery is used as the reference in subsequent comparisons between implants, the device 1 performs matching between the pre-surgery data 61 and the surgery data 62 by adjusting the coordinates of the surgery data 62 based on the pre-surgery data 61 during the matching process between the data 61 and 62. Even when the user performs fine adjustment of the matching result using the manipulator, the pre-surgery data 61 serves as the reference, and thus the device 1 performs fine adjustment by moving the position or rotating the orientation of the surgery data 62.

The matched data may be represented in at least one of a 3D image format or a 2D cross-sectional image format. In the case of matched data in a 2D cross-sectional image format, the device 1 for determining an implant placement position may provide basic cross-sectional images used for implant placement planning, such as cross-sections in three directions generated from 3D data—namely, axial, cross, and parallel cross-sections. The device 1 may receive a user operation signal to adjust the cross-sectional image and move the slice of the cross-section, allowing the user to review the matching results using the entire set of cross-sectional images.

FIG. 7 is a diagram showing an example of matching points between a radiopaque tool in surgery data and a radiopaque tool from the software library, according to an embodiment of the present invention.

Referring to FIGS. 1 and 7, the radiopaque tool 42 in the captured surgery data may become distorted or not be fully represented during the image acquisition process. The device 1 for determining an implant placement position may recognize the radiopaque tool 42 in the matched surgery data 4a and extract at least three points from among the topmost center point of the radiopaque tool 42, the center point of the stopper, and the bottommost center point. If a virtual line connecting the extracted points is not straight, the device may determine that distortion has occurred. The above is just one example of how distortion can be detected, and the method is not limited to this particular method.

If no distortion has occurred or the shape is fully represented, the device 1 for determining an implant placement position may proceed to the next step without performing the step of matching between the radiopaque tool 42 in the matched surgery data 4a and a radiopaque tool 5 from the library.

In contrast, if distortion has occurred or the shape is not fully represented, the device 1 for determining an implant placement position may match the shape of the radiopaque tool 42 represented in the matched surgery data 4a with the shape of the radiopaque tool 5 provided from the software library, as shown in FIG. 7. For example, the device 1 may automatically perform matching by detecting three points in each of the two radiopaque tools 42 and 5: the topmost center point 71a and 71b, one point 72a and 72b on the stopper extending across the drilling hole of the surgical guide, and a corresponding point 73a and 73b on the opposite side of the stopper. Although FIG. 7 shows the radiopaque tool 42 of the matched surgery data 4a in 2D cross-sectional form, the device 1 for determining an implant placement position may detect a matching region in a 3D shape. If the result of automatic matching is not satisfactory, a semi-automatic matching process is also possible, in which the user directly inputs a set of points (e.g., three points). The user may check the result of automatic or semi-automatic matching, and if necessary, modify the result using a matching adjustment manipulator.

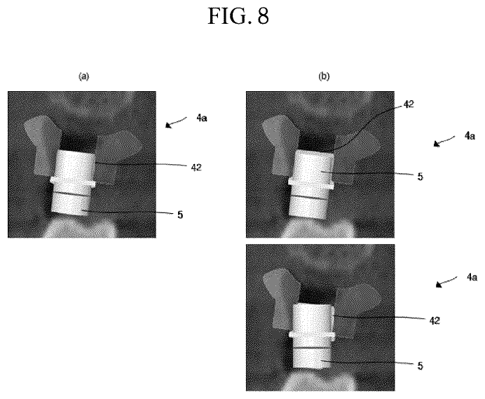

FIG. 8 is a diagram showing an image screen that provides a matching result of the radiopaque tool on the surgery data, according to an embodiment of the present invention.

Referring to FIGS. 1 and 8, the device 1 for determining an implant placement position provides a screen showing the matching result between the radiopaque tools 5 and 42 on the matched surgery data 4a, as illustrated in FIG. 8. The user may determine, via the matching result screen, whether the radiopaque tools 5 and 42 are properly matched (a), or whether a matching error has occurred (b). When a matching error has occurred (b), the device 1 for determining an implant placement position may receive two or more matching points directly from the user and perform an additional rematching process based on the input points.

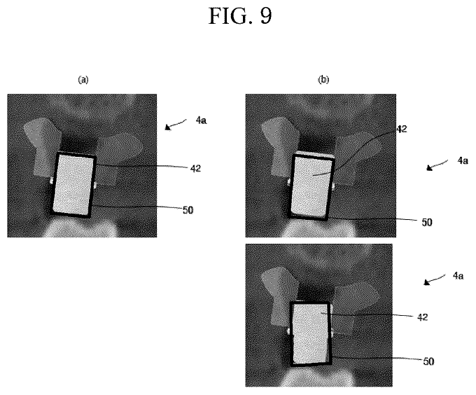

FIG. 9 is a diagram showing an image screen that provides the matching result of the radiopaque tool using a margin line, according to an embodiment of the present invention.

Referring to FIGS. 1, 8, and 9, the device 1 for determining an implant placement position may, as shown in FIG. 9, display a margin line 50 of the radiopaque tool 5 provided from the software library on the matched surgery data 4a in a 2D cross-sectional form so that it can be visually identified. This allows the user to determine whether the radiopaque tool 42 in the matched surgery data 4a matches the radiopaque tool 5 provided from the software library. If they match, the case corresponds to a successful match (a), and if they do not match, the case corresponds to a matching error (b). Furthermore, the device 1 for determining an implant placement position may provide a user interface that enables the user to check the matching result in a plurality of 2D cross-sectional images while automatically or manually adjusting the position at which a cross-section is generated. For example, in the case of surgery data in a 2D cross-sectional image format, the device 1 may provide basic cross-sectional images used for implant placement planning, such as cross sections in three directions generated from 3D data—namely, axial, cross, and parallel cross-sections. The device 1 may receive a user operation signal to adjust the cross-sectional image and move the slice of the cross-section, allowing the user to review the matching results using the entire set of cross-sectional images.

Since the coordinate information of the surgery data is transformed into matched surgery data through the matching process between the surgery data and the pre-surgery data, the device 1 for determining an implant placement position performs matching by adjusting the coordinate information of the radiopaque tool from the software library based on the radiopaque tool in the matched surgery data. Likewise, even when the user inputs a user operation signal via the manipulator to perform fine matching adjustment by changing the position of the radiopaque tool and rotating the direction, the device 1 maintains the reference coordinates of the matched surgery data and adjusts the position and orientation of the radiopaque tool provided from the software library to perform the fine matching adjustment.

During automatic matching of the radiopaque tool, when selecting a radiopaque tool from the software library, the device 1 for determining an implant placement position may select the type of the radiopaque tool from among narrow, regular, and wide types, based on the specification of the implant whose placement is planned before surgery for each tooth number. If the user inputs a user operation signal to change the specification of the radiopaque tool, the device 1 may activate the software library and allow the user to change the specification of the radiopaque tool for a tooth number selected by the user.

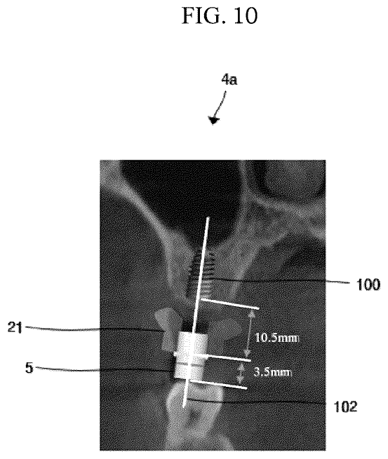

FIG. 10 is a diagram showing a screen for generating a virtual implant expected to be placed on the basis of the radiopaque tool according to an embodiment of the present invention.

Referring to FIGS. 1 and 10, once data matching between the radiopaque tools is completed, the device 1 for providing an implant placement position may compare the position of the implant planned before surgery with the implantation position of the virtual implant expected to be placed in the state in which the actual surgical guide is fastened, and provide the comparison result. To this end, the device 1 acquires coordinate information and shape of the implant from the pre-surgery data to identify the position of the implant planned before surgery, and may determine the expected implantation position of an implant 100 by using the matched radiopaque tool 5 from the matched surgery data 4a.

When the implant 100 is placed in the matched surgery data 4a, the uppermost part of the drilling hole 21 of the surgical guide is positioned at a location spaced a certain distance (e.g., 10.5 mm) upward along the reference axis (e.g., the long axis) of the implant 100, and the stopper of the radiopaque tool 5 is inserted up to the uppermost part of the drilling hole 21.

For example, if the thickness of the stopper portion of the radiopaque tool 5 is 0.5 mm and the height of the grid portion above the stopper is 3.0 mm, then, as shown in FIG. 10, the uppermost part of the virtual implant 100 expected to be placed is located 14.0 mm below the uppermost part of the radiopaque tool 5. When generating the shape of the virtual implant 100, the device 1 for providing an implant placement position may determine the central axis of the virtual implant 100 to be the same as the reference axis 102 (e.g., the long axis), which connects the uppermost and bottommost center points of the radiopaque tool 5. At this time, the specification of the virtually generated implant 100 may be obtained from the software library to match the specification of the implant that is planned to be placed for the corresponding tooth number during the surgery planning process.

FIG. 11 is a diagram showing (a) an image screen of an implant planned before surgery, (b) an image screen of a virtual implant expected to be placed based on surgery data, and (c) an image screen in which both implants are superimposed, according to an embodiment of the present invention.

Referring to FIGS. 1 and 11, the device 1 for providing an implant placement position may provide an image screen that compares the position and direction of the implant 20 planned using the pre-surgery data 2 with the position and direction of the virtual implant 100 expected to be placed in the matched surgery data 4a in the state in which the actual surgical guide is fastened in the patient's oral cavity. For example, as shown in FIG. 11, the device 1 may provide a superimposed image screen 110 of the shape of the implant 20 planned before surgery and the shape of the virtual implant 100 expected to be placed in the matched surgery data 4a.

The device 1 for providing an implant placement position may also display a safety region 112 of the implant 20 planned before surgery on the superimposed image screen 110 of the two implants. The safety region 112, which is an area set at a preset distance from the implant 20 planned before surgery, is set by taking into consideration errors that may occur during the implant implantation process and may be set such that the user can adjust the range of the safety region 112.

FIG. 12 is a drawing showing an image screen that provides error information between a position of an implant planned before surgery and a position of a virtual implant expected to be placed according to one embodiment of the present invention.

Referring to FIGS. 1 and 12, the device 1 for providing an implant placement position may measure an angle between the reference axis of the implant 20 planned before surgery and the reference axis of the virtual implant 100 expected to be placed, and provide the angle difference. In this case, if the angle difference between the reference axis of the implant planned before surgery and the reference axis of the virtual implant exceeds a preset angle, the device 1 may determine that an error has occurred and provide error information.

Furthermore, the device 1 may provide a measurement interface in the superimposed image screen 110 of the implants, allowing the user to check error information between the two implants 20 and 100. In FIG. 12, an example is illustrated in which four points are input by the user through the measurement interface to generate two lines and the angle between the two lines (e.g., 4.5 degrees) is measured. Specifically, the device 1 may generate a center axis line for each of the two implants 20 and 100 and measure and provide the angle between the two center axis lines.

If the user has set an automatic measurement region through user settings, for example, selecting “measure coordinate information of the lowermost part of the implant,” the device 1 for providing an implant placement position may activate the measurement interface that automatically detects a deviation of the position of the lowermost point of the virtual implant 100 from the position of the implant 20 planned before surgery in the superimposed image screen 110.

As another example, when the user setting is configured to check the implantation direction error based on the reference axes (e.g., long axes) of each implant 20 and 100, the device 1 for providing an implant placement position may automatically measure the angle (e.g., 4.5 degrees) between the reference axis of the implant 20 planned before surgery and the reference axis of the virtual implant 100, and provide the angle deviation information.

FIG. 13 is a diagram showing an image screen that provides warning information when the virtual implant expected to be placed deviates from the safety region of the implant planned before surgery, according to an embodiment of the present invention.

Referring to FIGS. 1 and 13, when the virtual implant 100 expected to be placed deviates from the safety region 112 of the implant 20 planned before surgery, the device 1 for providing an implant placement position may provide warning information indicating such a deviation. For example, as shown in FIG. 13, when the virtual implant 100 is out of the safety region 112, the device 1 may display the virtual implant 100 in a distinguishable color on the superimposed image screen 110. Through the warning information, the user may confirm that the virtual implant 100 expected to be placed has deviated from the safety region 112 of the implant 20 planned before surgery.

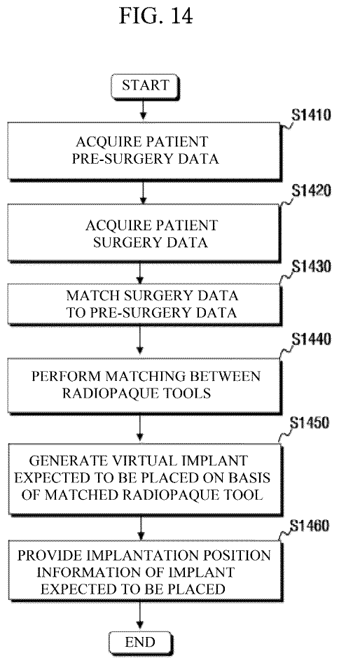

FIG. 14 is a flowchart illustrating a method for providing an implant placement position according to an embodiment of the present invention.

Referring to FIGS. 1 and 14, in step S1410, the device 1 for providing an implant placement position acquires pre-surgery data of a patient to be used for surgery planning, including the design of a virtual guide shape. The device 1 may determine the placement position of an object, including a virtual implant, from the acquired pre-surgery data of the patient and then determine the position of a drilling hole based on the determined placement position of the implant. The device 1 may then design and output a virtual guide shape including the determined drilling hole.

Next, in step S1420, the device 1 for providing an implant placement position acquires surgery data by imaging the patient's oral cavity, the surgery data including a radiopaque tool and a surgical guide. In step S1420 of acquiring surgery data, the device 1 may fasten an actual radiopaque tool into the drilling hole of an actual surgical guide that is mounted in the patient's oral cavity, and then photograph the oral cavity to acquire the corresponding surgery data.

Subsequently, in step S1430, the device 1 for providing an implant placement position matches the surgery data to the pre-surgery data. In step S1430 of matching the pre-surgery data and the surgery data, the device 1 may perform matching by adjusting the position and orientation of the surgery data with respect to the pre-surgery data. After matching the pre-surgery data and the surgery data, the device 1 may receive a user operation signal via a manipulator to perform fine-adjustment of the matching result.

Next, in step S1440, the device 1 for providing an implant placement position may match the radiopaque tool provided from the software library, on the basis of the radiopaque tool represented in the matched surgery data. For the purpose of matching the radiopaque tools, the device 1 may select a radiopaque tool from the software library to be matched with the radiopaque tool in the surgery data. At this time, the device 1 may select the type of the radiopaque tool based on the specification of the implant whose placement is planned before surgery for each tooth number.

In step S1440 of matching radiopaque tools, the device 1 for determining an implant placement position may match the radiopaque tools by adjusting the position and orientation of the radiopaque tool provided from the software library with respect to the radiopaque tool represented in the matched surgery data.

Also in step S1440, the device 1 may extract and match points for the two radiopaque tools, such as the topmost center point, a point on a stopper extending across the drilling hole of the surgical guide, and a corresponding point on the opposite side of the stopper.

Furthermore, the device 1 may provide, as an image screen, a matching result that indicates whether the shape of the radiopaque tool in the surgery data matches the margin line of the radiopaque tool provided from the software library. At this time, the device 1 may provide the matching result while varying the position at which a cross-section is generated within the 2D cross-sectional image screen that shows the matching result between the two radiopaque tools.

Next, in step S1450, the device 1 for providing an implant placement position generates a virtual implant expected to be placed on the basis of the matched radiopaque tool.

Then, in step S1460, the device 1 provides placement position information of the implant expected to be placed.

In step S1460, the device 1 may provide an image screen comparing the position and orientation of the implant whose placement is ( [symbol] as) planned using the pre-surgery data with those of the virtual implant expected to be placed in the state in which the actual surgical guide is fastened in the patient's oral cavity.

For example, in the superimposed image screen of the implants, the device 1 may provide error information indicating how much the position of the virtual implant expected to be placed deviates from the position of the implant whose placement is planned before surgery.

At this time, the device 1 may provide a measurement interface that allows the user to check the error information between the implant whose placement is planned before surgery and the virtual implant expected to be placed.

As another example, the device 1 may measure the angle between the reference axis of the implant whose placement is planned before surgery and the reference axis of the virtual implant expected to be placed, and provide angle deviation information.

As yet another example, if the virtual implant expected to be placed deviates from the safety region of the implant whose placement is planned before surgery, the device 1 may provide warning information indicating such a deviation.

So far, the present invention has been described with reference to embodiments thereof. It will be understood by those skilled in the art that various changes in form and details may be made therein without departing from the essential features of the present invention. Therefore, the disclosed embodiments should be considered in descriptive sense only and not for purposes of limitation. The scope of the present invention should be defined not by the detailed description but by the appended claims, and all differences falling within a scope equivalent to the claims should be construed as being encompassed by the present invention.

1. A method for determining an implant placement position using a device for determining an implant placement position, the method comprising the steps of:
acquiring pre-surgery data of a patient including design of a virtual guide shape to be used for surgical planning;
acquiring surgery data including a radiopaque tool and a surgical guide by photographing an oral cavity of the patient;
matching the surgery data to the pre-surgery data;
generating a virtual implant expected to be placed on the basis of a radiopaque tool in the matched surgery data; and
providing placement position information of an implant expected to be placed.

2. The method of claim 1, further comprising the step of determining a placement position of an object including the virtual implant from the pre-surgery data of the patient and determining a position of a drilling hole based on the determined placement position of the implant, followed by designing and outputting a virtual guide shape including the determined drilling hole.

3. The method of claim 1, wherein the step of acquiring the surgery data comprises fastening an actual radiopaque tool into a drilling hole of an actual surgical guide that is fastened within the oral cavity of the patient, and then photographing the oral cavity to acquire the surgery data, and
the step of matching the surgery data comprises matching the surgery data on the basis of the pre-surgery data by adjusting position and orientation of the surgery data to align with position and orientation of the pre-surgery data.

4. The method of claim 1, further comprising the step of, after matching the pre-surgery data and the surgery data, performing fine-adjustment of a matching result using a manipulator.

5. The method of claim 1, further comprising the step of, after matching the pre-surgery data and the surgery data, matching a radiopaque tool provided from a software library on the basis of the radiopaque tool represented in the matched surgery data.

6. The method of claim 5, further comprising the step of selecting a radiopaque tool from the library to be matched with the radiopaque tool of the surgery data,
wherein a type of radiopaque tool is selected based on a specification of an implant whose placement is planned before surgery for each tooth number.

7. The method of claim 5, wherein the step of matching the radiopaque tools comprises matching the radiopaque tools by adjusting position and orientation of the radiopaque tool provided from the software library with respect to the radiopaque tool represented in the matched surgery data.

8. The method of claim 5, wherein the step of matching the radiopaque tools comprises extracting and matching points for the two radiopaque tools, the points including a topmost center point, a point on a stopper extending across a drilling hole of the surgical guide, and a corresponding point on an opposite side of the stopper

9. The method of claim 5, further comprising the step of providing, as an image screen, a matching result indicating whether a shape of the radiopaque tool in the matched surgery data matches a margin line of the radiopaque tool provided from the software library.

10. The method of claim 9, wherein the step of providing the matching result as the image screen comprises providing the matching result while varying a position at which a cross-section is generated within a two-dimensional (2D) cross-sectional image screen that provides the matching result between the two radiopaque tools.

11. The method of claim 1, further comprising the step of providing an image screen comparing position and orientation of an implant whose placement is planned using the pre-surgery data with those of the virtual implant expected to be placed in a state in which an actual surgical guide is fastened in the patient's oral cavity.

12. The method of claim 11, wherein the step of providing the image screen comprises measuring, in a superimposed image screen where the implant whose placement is planned and the virtual implant are overlapped, an angle between a reference axis of the virtual implant expected to be placed and a reference axis of the implant whose placement is planned before surgery, and providing an angle difference, wherein, when the angle difference between the reference axis of the implant whose placement is planned before surgery and the reference axis of the virtual implant expected to be placed is greater than a preset angle, it is determined that an error has occurred, and error information is provided.

13. The method of claim 12, wherein the step of providing the image screen comprises providing a measurement interface that allows the user to check the error information between the position of the implant whose placement is planned before surgery and the position of the virtual implant expected to be placed.

14. The method of claim 11, wherein the step of providing the image screen comprises measuring an angle between a reference axis of the implant whose placement is planned before surgery and a reference axis of the virtual implant expected to be placed and providing angle deviation information.

15. The method of claim 11, wherein the step of providing the image screen comprises, when the virtual implant expected to be placed deviates from a safety region of the implant whose placement is planned before surgery, providing warning information indicating this deviation.

16. A device for determining an implant placement position, comprising:
a data acquisition unit configured to acquire pre-surgery data of a patient including design of a virtual guide shape to be used for surgical planning, and acquire surgery data including a radiopaque tool and a surgical guide by photographing an oral cavity of the patient;
a control unit configured to match the surgery data to the pre-surgery data and generate a virtual implant expected to be placed on the basis of a radiopaque tool in the matched surgery data; and
an output unit configured to output placement position information of an implant expected to be placed.

17. The device of claim 16, wherein the control unit is configured to display, via the output unit, an image screen comparing position and orientation of an implant whose placement is planned using the pre-surgery data with those of the virtual implant expected to be placed in a state in which the patient is wearing an actual surgical guide.

18. The device of claim 16, wherein the control unit is configured to, after matching the pre-surgery data and the surgery data, match a radiopaque tool provided from a software library on the basis of the radiopaque tool represented in the matched surgical data.
